(19) United States
(12) Patent Application Publication
(10) Pub. No.: US 2026/0090687 A1
(43) Pub. Date: Apr. 2, 2026

(54) AUTOMATIC VACUUM SYSTEM FOR PREVENTING CLOGGED AC DRAIN LINES

(71) Applicant: Andrew Everett ERICKSON, Kamuela, HI (US)

(72) Inventor: Andrew Everett ERICKSON, Kamuela, HI (US)

(21) Appl. No.: 19/338,856

(22) Filed: Sep. 24, 2025

Related U.S. Application Data
(60) Provisional application No. 63/700,470, filed on Sep. 27, 2024.

(51) Int. Cl. A47L 7/00; A47L 9/28; A61L 2/10; F24F 13/22

(52) U.S. Cl. CPC A47L 7/0004; A47L 7/0061; A47L 9/2842; A61L 2/10; F24F 13/222; A61L 2202/11; A61L 2202/17; B08B 2209/032; F24F 2013/227; F24F 2221/22

(57) ABSTRACT

An HVAC condensation line cleaning system, having: a housing with an internal chamber; an HVAC drain line draining into the internal chamber; a timed vacuum assembly that generates a vacuum in the internal chamber; and a check valve on the housing that: (a) opens permitting fluid to drain from the HVAC drain line out of the housing when the vacuum assembly is turned off, and (b) closes to generate suction in the HVAC drain line, thereby sucking contents of HVAC drain line into the internal chamber when the vacuum assembly is turned on.

### RELATED APPLICATION

The present invention claims priority to U.S. Provisional Patent Application Ser. No. 63/700,470, of same title, filed Sep. 27, 2024, the entire disclosure of which is incorporated herein by reference in its entirety for all purposes.

### TECHNICAL FIELD

The present invention relates to the maintenance of HVAC Systems. Specifically, the present invention is an automated vacuum system that regularly removes microbial buildup, dirt, and debris from HVAC condensation lines.

### BACKGROUND OF THE INVENTION

Air conditioning systems produce condensation as part of their cooling process. This condensation is drained out through an HVAC condensation or drain line. Over time, these lines can become clogged with microbial growth, dirt, dust, and air particulates. When the line gets blocked, water backs up and overflows from the AC unit, which can cause significant water damage. Clogged drain lines are one of the primary reasons for expensive and disruptive emergency service calls. During an emergency service call, the most common method to clear a clogged drain line is to attach a shop vacuum to the end of the drain line and manually vacuum out the clog. Emergency service calls are often disruptive and result in large service fees. Current solutions for maintaining clear drain lines include the following:

First, pressurized air or water flush systems as described by Plexaire in U.S. Pat. No. 8,961,708 and Safe-T-Flush in its U.S. Pat. No. 8,978,690. These pressurized systems are designed to remove solid debris by pushing air or water through the condensation line to force out blockages. Although effective at clearing both microbial buildup and physical debris, such as dirt and dust, they come with significant drawbacks. The major drawback is risk of pipe damage. This is because the force exerted by pressurized air or water systems can damage condensation lines, particularly in older or more delicate systems. Ruptured pipes can lead to costly repairs. Furthermore, modern split AC systems are often mounted directly to walls, thus offering limited space for installing flushing or cleaning devices near the AC unit.

Second, chemical solutions (e.g., the use of Antimicrobial Agents or Soaps) such as described by Iflo US in its U.S. Pat. No. 11,761,675. These chemical solution products aim to reduce microbial growth inside the drain line. Unfortunately, they are less effective at preventing clogs caused by solid debris like dirt, dust, and air particulates. In addition, chemical solutions may slow down microbial growth, but they do nothing to address the accumulation of physical debris, which is a common cause of clogs. Chemical solutions are also known to cause damage to delicate drain lines due to their caustic and corrosive nature. Similar to pressurized water and gas devices, they provide limited physical access and these space restraints often prevent the use of these systems.

Third, U.S. Pat. No. 8,535,453 to Gonzalez discloses a condensation line cleaning system that uses a timed vacuum pump to periodically draw water out of the condensation line. When the vacuum is off, the condensation simply drains down through the still blades of its impeller pump and out the bottom of the device. A major limitation with this system is that if the water draining out freezes, it can lock the blades of the impeller pump in place. This results in damage to the pump if it is frozen in place and then turned on. In addition, the Gonzalez system has water draining through/over impellers have narrow passageways that clog easily. Such constantly wet impellers allow bacteria, slime, and growths to build up, locking the impellers. This results in exactly the same problem as with ice. Each of Gonzalez's pump housing and discharge pipe are narrow, so sludge and buildup can clog or damage the impellers.

It would instead be desirable to provide a system in which condensation does not simply drain through the blades of the impeller pump when the pump is off. Also, when the pump is turned on and operating properly, water is simply forced across the impeller blades at high pressures. It would instead be desirable to avoid water being forced across the impeller blades at high pressures. It would also be desirable to provide a system in which only air passes through the pump to avoid the pump jamming. It would also be desirable to provide UV sterilization (which is not possible in the Gonzalez's system since it does not have a housing in which the discharge can be sealed and sterilized with UV light).

A final approach is simply performing expensive routine maintenance. Unfortunately, such regular maintenance to clear condensation drain lines often needs to be performed by professional service techs. HVAC technicians arrive and typically just use shop vacuums to manually remove debris from the drain line. This can be both time-consuming and costly. These service calls are necessary to prevent water damage but can add up quickly, making routine maintenance expensive for homeowners and businesses.

What is instead desired is a simple, cost effective, and preferably automatic way to address these above problems. As will be shown, the present system provides such a solution.

### SUMMARY OF THE INVENTION

The present invention provides an automatic vacuum system that attaches to the end of an HVAC condensation drain line to prevent clogs and reduce the need for emergency service calls. The present system operates on a schedule and uses suction to remove both microbial buildup and solid debris, including dirt and dust, from the condensation line. By preventing clogs and overflow, the present system addresses one of the leading causes of costly AC service calls. Advantageously, the vacuum is designed to work with both central attic systems and split systems mounted on walls, where space constraints make traditional flushing devices impractical. In preferred aspects, the present system is simply installed at the termination point of the HVAC condensation line, often outside the building. This has the advantage of providing easy access and efficient clog prevention.

In preferred aspects, the present system provides a system for automatically cleaning an HVAC condensation line, comprising: (a) a housing having an internal chamber; (b) an HVAC drain line configured to drain into the internal chamber of the housing; (c) a vacuum assembly configured to generate a vacuum in the internal chamber of the housing; (d) a timing system that periodically activates the vacuum assembly; and (e) a check valve on the housing, wherein: (i) the check valve opens permitting fluid to drain from the HVAC drain line through the housing and out of the housing when the vacuum assembly is turned off, and (ii) the check valve closes generating suction in the HVAC drain line, thereby sucking contents of HVAC drain line into the internal chamber of the housing when the vacuum assembly is turned on.

The HVAC drain line is configured to be connected to a condensate drain line of an HVAC system. In operation, the check valve opens under the force of gravity and closes when the vacuum system generates a vacuum in the internal chamber of the housing, thereby lifting the check valve into a closed position. In operation, the timing system may be programed to activate the vacuum assembly on a daily or weekly basis, or at some other time interval or schedule. In addition, the operation schedule can optionally be changed depending upon the season or weather patterns.

In further optional aspects, the system further comprises a disinfecting UV light in the internal chamber of the housing. This disinfecting UV light is preferably turned on when the vacuum system is turned on. As a result, the contents of the internal chamber of the housing can be disinfected prior to the contents of the internal chamber being disposed to the environment.

The present system preferably has a pump in which water or debris never pass over the pump's impeller blades. As a result, the present system provides a large sealed chamber in which debris and water can be disinfected. Optionally, this large sealed chamber can be placed in its own detachable housing.

A first advantage of the present system is that it provides effective removal of both microbial buildup and the buildup of physical debris. While existing pressurized air and water systems can effectively clear solid debris, they come with the risk of damaging pipes. In contrast, the present system gently removes both microbial growth and debris without applying excessive force, thereby minimizing the risk of pipe damage.

A second advantage of the present system is that it prevents costly emergency service calls. Since clogged drain lines are a common reason for AC service calls, the present automatic vacuum system offers a proactive solution to maintain clear lines, thereby reducing the need for emergency maintenance. By automatically clearing the HVAC line on a regular schedule, the present system eliminates the need for manual intervention (such as using a shop vacuum during service visits).

A third advantage of the present system is that it is suitable for split AC systems. Specifically, common wall-mounted HVAC split systems offer limited space for installing traditional maintenance tools or flushing devices. In contrast, the present automatic vacuum system is designed to be installed at the end of the condensation line, typically at an outdoors location, where it can operate without taking up space near the AC unit itself.

In summary, the present invention is an automatic vacuum system that connects to the end of a HVAC condensation drain line. It runs on a set schedule to remove microbial buildup, dirt, dust, and other particles, preventing clogs and water overflow. Unlike devices that utilize pressurized air or water, which can damage pipes, or chemical treatments that don't clear solid debris, this vacuum provides a safer and more effective way to keep drain lines clear and reduce expensive emergency service calls.

Advantageously, the condensation that enters and then drains out of the internal chamber of the housing leaves at the bottom of the housing (i.e.: through the bottom check valve). As such, the condensation does not pass through the blades of the pump itself. This is because the vacuum pump is at the top of the housing and the check valve is at the bottom of the housing. This is particularly advantageous in that should the water in the housing ever freeze, it could not damage the blades of the vacuum pump above. As a result, the risk of the pump blades freezing into a locked position is completely avoided.

### BRIEF DESCRIPTION OF DRAWINGS

FIGS. 1 to 4 show detailed view of the housing, vacuum assembly and check valve of the present system showing its operation. FIG. 5 shows a schematic of the present system installed at the end of an AC condensation drain line, as follows.

FIG. 1 is a side perspective view of the present system.

FIG. 2 is a side perspective view of the present system with the three sections of the device body “exploded” to show the interior components.

FIG. 3 is a side perspective view of the present system with a cut away of the front wall removed to show the interior components.

FIG. 4 is a bottom exploded perspective view of the present system showing the check valve and its associated plunger.

FIG. 5 is a schematic illustration of the present system installed at the end of an AC condensation drain line.

### DETAILED DESCRIPTION OF THE INVENTION

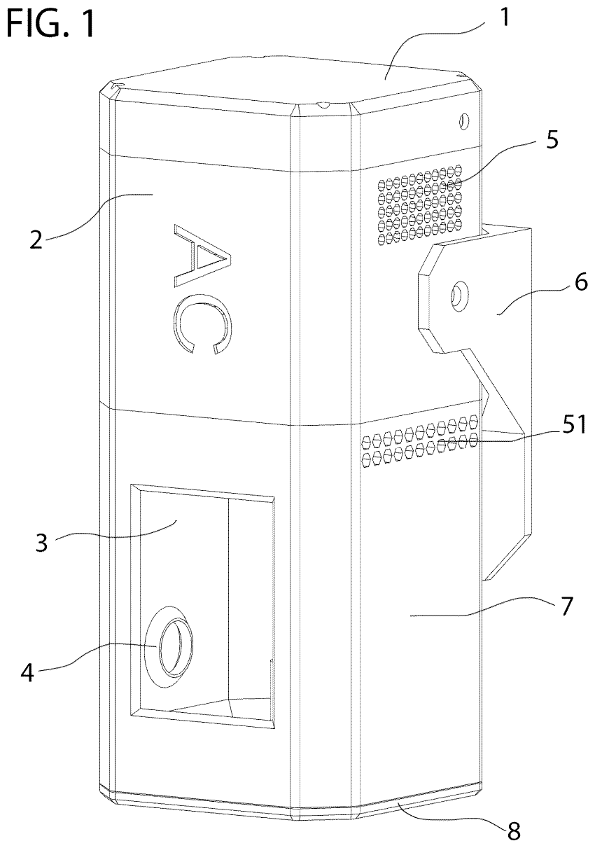

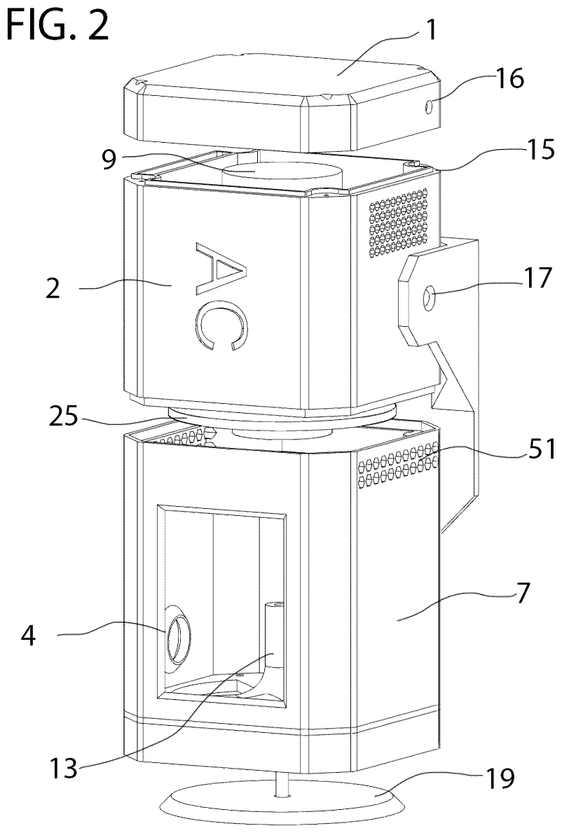

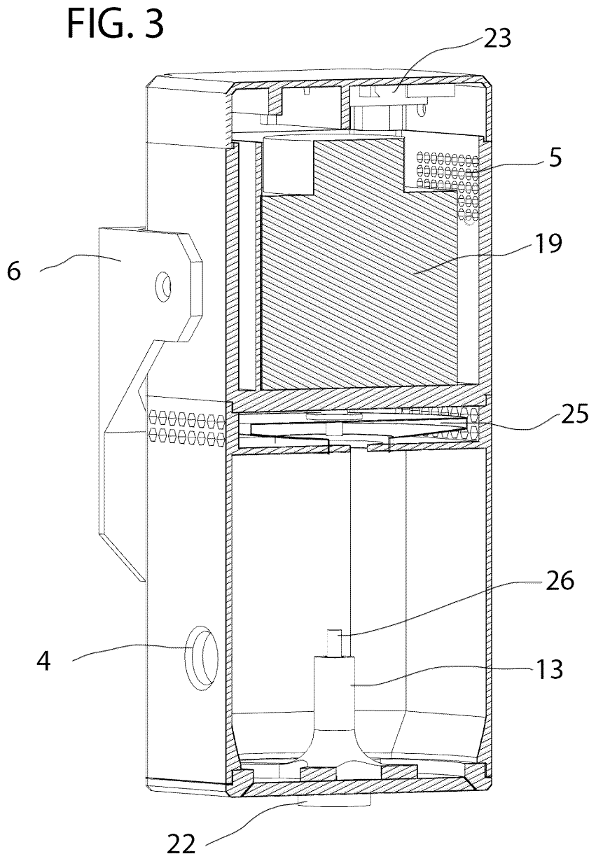

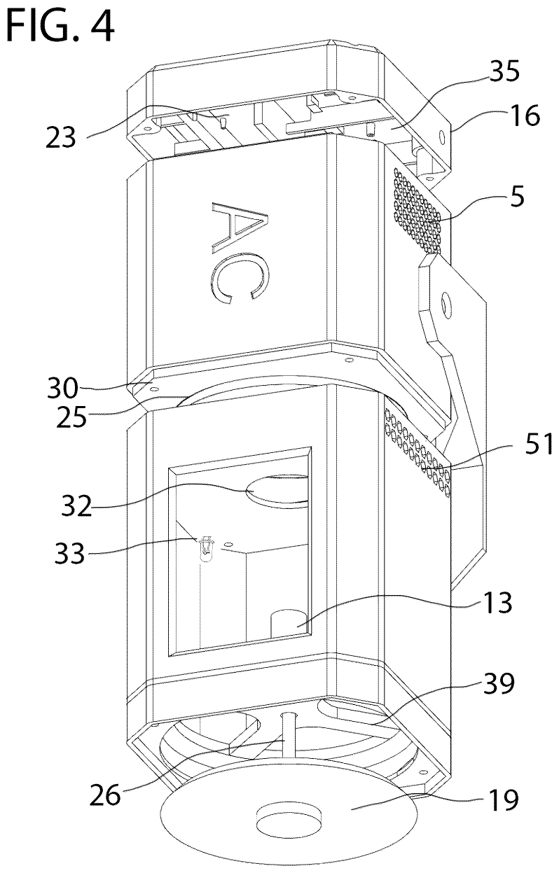

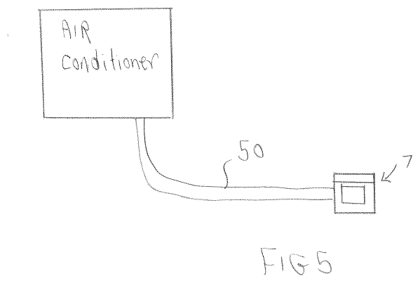

As seen in FIGS. 1 to 4, the present system provides a system for automatically cleaning an HVAC condensation line. The system comprises: a housing 7 having an internal chamber 3; an HVAC drain line (50 in FIG. 5) configured to drain through drain line input 4 into the internal chamber 3 of the housing 7; a vacuum assembly 2 configured to generate a vacuum in internal chamber 3 of housing 7. A timing system may be housed under top lid 1 that periodically activates the vacuum assembly 2. A check valve 19 is also provided on the bottom of housing 7. Housing 7 may be mounted to a wall by bracket 6. A convenient bolt hole 17 is provided for this mounting.

In operation, the check valve 19 opens permitting fluid to drain from the HVAC drain line 50 down through housing 7 and out of the bottom of the housing when the vacuum assembly 2 is turned off. Conversely, check valve 19 closes generating suction in the HVAC drain line 50, thereby sucking contents of an associated HVAC drain line into the internal chamber 3 of housing 7 when the vacuum assembly 2 is turned on. When the vacuum assembly 2 is turned on and its motor 9 is activated centrifugal blower wheel 25 rotates, pulling air through its center and expelling it in a radial direction so that air is drawn out of the top of inner chamber 3 through fan intake hole 32 and is expelled through vent holes 51. When a vacuum is created in internal chamber 3, plunger 26 is pulled upwardly in plunger guide 13, thereby closing the bottom opening into internal chamber 3. Plunger 26 is supported at its bottom by plunger support 22, and centered by supports 39. Simply put, check valve 19 opens under the force of gravity and closes when the vacuum system 2 generates a vacuum in the internal chamber 3 of the housing 7, thereby lifting the check valve 19 into a closed position. This vacuum will then suck any blockage out drain line 50, placing the blockage into internal chamber 3. An optional disinfecting UV light 33 can be placed in internal chamber 3 of the housing, and this disinfecting UV light can be turned on when the vacuum system is turned on. Preferably, the vacuum assembly operates on a low-power motor, providing sufficient suction to maintain a clear drain line while minimizing energy consumption. Top portion 1 preferably houses the controller and power supply. Air vents 5 provide ventilation for a Printed Circuit Board and associated power supply within the top of the device.

Optionally, the chamber 3 in the housing can be provided in its own detachable housing.

Additional features may include a hole 16 for a power cord, a bracket 23 for the PCB board WIFI and Bluetooth controls (for example a standard smart appliance WIFI controller and IOS app), UV light 33 and a bracket 35 for a power supply. A removable housing bottom 8 can be used to permit the bottom of the device to be removed and cleaned periodically. A top ridge 15 can help provide a watertight seal for the device, and a bottom ridge 30 can be included. Ridges 15 and 30 may be used for positioning gaskets to provide a watertight seal.

In preferred aspects, the timing system is programed to activate the vacuum assembly on a daily or weekly basis (or some other schedule which may be varied per the seasons and weather). The vacuum operates on a programmed schedule, turning on for a set amount of time, such as once every 24 hours, to generate suction. When the vacuum activates, it creates suction that causes the check valve 19 at the bottom of the housing to close. This check valve 19 prevents any backflow, allowing the vacuum to direct its suction force exclusively toward the condensation line 50. As the suction removes accumulated water, microbial growth, dirt, and debris from the condensation line, these materials are drawn into the holding compartment (internal chamber 3) of the vacuum device. Once the vacuum shuts off, the suction force inside the holding compartment stops, and the check valve reopens, utilizing gravity to fall back into its resting position. With the check valve open, the contents of the holding compartment, including any collected water and debris, are released by gravity through the bottom of the device. This mechanism ensures that the device effectively vacuums out blockages from the condensation line while also automatically emptying itself, allowing for continuous operation without the need for manual intervention or maintenance. The simple, gravity-assisted design of the check valve eliminates the need for complex moving parts or filters, making the system highly reliable and low maintenance. When the present device is inactive, the check valve advantageously remains in the open position, allowing condensation to flow freely through the drain line and out the bottom of the device.

One advantage of the present system is that it is designed to remove both microbial buildup and solid debris, such as dirt, dust, and air particulates, without the use of pressurized air or water, thereby preventing damage to the drain line. Another advantage of the present system is that the check valve opens due to gravity once suction stops, allowing the holding compartment to empty automatically. All of this is done while keeping the condensation draining out of the housing away from the vacuum pump blades (at the top of the housing). As a result, this invention provides an efficient, hands-off solution to the problem of clogged condensation drain lines in air conditioning systems, particularly split systems. By using a scheduled vacuum system, it offers a safer, more reliable alternative to pressurized air or water systems while addressing the limitations of chemical solutions. The automatic vacuum prevents clogs that lead to costly service calls, ensuring long-term AC performance and reducing maintenance needs.

What is claimed is:

1. A system for automatically cleaning an HVAC condensation line, comprising:
a housing having an internal chamber;
an HVAC drain line configured to drain into the internal chamber of the housing;
a vacuum assembly configured to generate a vacuum in the internal chamber of the housing;
a timing system that periodically activates the vacuum assembly; and
a check valve on the housing, wherein:
the check valve opens permitting fluid to drain from the HVAC drain line through the housing and out of the housing when the vacuum assembly is turned off, and
the check valve closes generating suction in the HVAC drain line, thereby sucking contents of HVAC drain line into the internal chamber of the housing when the vacuum assembly is turned on.

2. The system of claim 1, wherein the HVAC drain line is configured to be connected to a condensate drain line of an HVAC system.

3. The system of claim 2, wherein the HVAC system is an air conditioner.

4. The system of claim 1, wherein the check valve opens under the force of gravity and closes when the vacuum system generates a vacuum in the internal chamber of the housing, thereby lifting the check valve into a closed position.

5. The system of claim 1, further comprising:
a disinfecting UV light in the internal chamber of the housing, wherein the disinfecting UV light is turned on when the vacuum system is turned on.

6. The system of claim 1, wherein the timing system is programed to activate the vacuum assembly on a daily or weekly basis.

7. The system of claim 1, wherein the internal chamber of the housing is detachable from the housing.
